(19) United States
(12) Patent Application Publication
(10) Pub. No.: US 2025/0312353 A1
(43) Pub. Date: Oct. 9, 2025

(54) DRUG REPURPOSING FOR TREATMENT OF CONGENITAL VASCULAR MALFORMATIONS

(71) Applicant: University of South Carolina, Columbia, SC (US)

(72) Inventors: Vi Nguyen, Columbia, SC (US); Wenbin Tan, Columbia, SC (US); Chao Gao, Columbia, SC (US); Anil Goud Jegga, Cincinnati, OH (US); Yunguan Wang, Cincinnati, OH (US); Sudhir Ghandikota, Cincinnati, OH (US); Marcelo Hochman, Mt. Pleasant, SC (US)

(73) Assignee: University of South Carolina, Columbia, SC (US)

(21) Appl. No.: 19/044,791

(22) Filed: Feb. 4, 2025

Related U.S. Application Data
(60) Provisional application No. 63/574,435, filed on Apr. 4, 2024.

(51) Int. Cl. A61K 31/5415; A61K 31/07; A61K 31/36; A61K 31/4422; A61K 31/4535; A61K 31/47; A61K 31/4745; A61K 31/4985; A61K 31/506; A61K 31/5386; G16B 5/30; G16B 40/20

(52) U.S. Cl. CPC A61K 31/5415; A61K 31/07; A61K 31/36; A61K 31/4422; A61K 31/4535; A61K 31/47; A61K 31/4745; A61K 31/4985; A61K 31/506; A61K 31/5386; G16B 5/30; G16B 40/20

(57) ABSTRACT

Described herein is a data-driven computational approach to identify candidate molecules and characterize them using prior knowledge of the Port Wine Birthmark—associated biological processes and pathways. The signature-based connectivity map (CMap)47 computational drug discovery module took a collection of transcriptomic signatures (DEGs) as input and outputs a list of ranked drugs predicted to consistently reverse the input gene signature(s) as input.

### GOVERNMENT LICENSE RIGHTS

This invention was made with government support under HT9425-23-1-0008 awarded by the Defense Health Agency, Medical Research and Development Branch, and R01 AR073172 awarded by the National Institutes of Health. The government has certain rights in the invention.

### TECHNICAL FIELD

The subject matter disclosed herein is generally directed to data-driven computational approach to identify candidate molecules and characterize them using prior knowledge of the Port Wine Birthmark—associated biological processes and pathways. The signature-based connectivity map (CMap)47 computational drug discovery module took a collection of transcriptomic signatures (DEGs) as input and outputs a list of ranked drugs predicted to consistently reverse the input gene signature(s) as input.

### BACKGROUND

Drug repurposing is a strategy to identify new applications for an existing drug beyond its original medical indication. These compounds have available preclinical and clinical data thus are de-risked with the advantages of low costs and short timelines. It will particularly benefit patients with rare and neglected or uncommon diseases, including Port Wine Birthmarks (PWB) that are largely ignored by the pharmaceutical industries and currently lack effective treatments.

The inadequate clinical outcomes of current energy-based treatment modalities for PWB need paradigm-shifting therapeutics, such as targeting causal mechanistic signaling pathways to reduce lesional vascular phenotypes. Accordingly, it is an object of the present disclosure to provide a strong rationale for introducing such a modality.

Citation or identification of any document in this application is not an admission that such a document is available as prior art to the present disclosure.

### SUMMARY

The above objectives are accomplished according to the present disclosure by providing in one instance a method for repurposing an existing drug to treat Port Wine Birthmarks. The method may include employing at least one data-driven computational module to identify and characterize at least one existing drug candidate molecule based on at least one effect of the at least one existing drug candidate on at least one Port Wine Birthmark biological process, inputting at least one transcriptomic signature as at least one input; and outputting at least one existing drug, wherein the at least one existing drug is selected to reverse the at least one transcriptomic signature used as the at least one input. Further, an original medical indication of the existing drug may not include treatment of Port Wine Birthmarks. Additionally, the existing drug may target at least one causal mechanistic signaling pathway to reduce at least one lesional vascular phenotype. Still further, the at least one causal mechanistic signaling pathway may comprise at least one aberrant Hippo/Wnt pathway. Moreover, the existing drug is selected from acepromazine, bedaquiline, calmidazolium, celastrol, cilomilast, ciprofibrate, clopidogrel, epothilone, evodiamine, imatinib, irinotecan, isoeugenol, isotretinoin, linifanib, nifedipine, raloxifene, retinol, scopolamine, serdemetan, sitagliptin, topiramate, xanthohumol, Y-27632 and/or combinations of the above. Again further, the at least one data-driven computational module comprises a signature-based connectivity map (CMap)47 computational drug discovery module. Still yet again, multiple existing drugs may be provided as an output. Again, the multiple existing drugs may be provided as an output and may be ranked in order of efficacy with respect to reversing the at least one transcriptomic signature used as the at least one input. Further still again, an encoder-decoder framework may be used to rank the multiple existing drugs provided as the output via presenting at least one predictive drug-target interaction model. Even further, at least one functional enrichment analysis may be conducted on the at least one existing drug candidate to characterize the at least one existing drug candidate with respect to blood vessel development, EC migration, blood vessel diameter maintenance, and Wnt regulation.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIGS. 1A-1C show at: (A) Transcriptome profiles of PWB iPSCs and ECs versus normal ones (n=6-8 for each group). RNA-seq data plot showing significantly upregulated (red dots) or downregulated (blue dots) genes (FDR<0.01). Black dots: genes without expression changes. (B) KEGG analysis of RNA-seq DEGs showing significantly dysregulated pathways overlapped in both PWB iPSCs and ECs as compared with normal ones. −log (P) was plotted. (C) Enriched DEGs associated with Hippo and Wnt signalosomes in PWB iPSC-derived EC from bulk RNA-seq data. (D) The coverage and intensity of called peaks showing more chromatin accessibility in the promoters of WNT5B and NKD1 genes in PWB ECs as compared to normal ECs using ATAC-seq.

FIG. 2 shows one embodiment of a workflow of the current disclosure.

FIG. 3 shows a listing of compounds common to target both Wnt and Hippo signalizing pathways from ECs and iPSCs of PWB.

FIG. 4 shows a schematic representation of encoder-decoder framework to discover and prioritize drug target interactions.

FIG. 5 shows a network representation of deep learning-based drug-target predictions for bedaquiline and enriched biological processes.

FIGS. 6A-6D show Table 1—compounds for effect against PWB.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

As used herein, “dose,” “unit dose,” or “dosage” can refer to physically discrete units suitable for use in a subject, each unit containing a predetermined quantity of an active compound and/or a pharmaceutical formulation thereof calculated to produce the desired response or responses in association with its administration.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof. “A polymer” can be can be a three-dimensional network (e.g. the repeat units are linked together left and right, front and back, up and down), a two-dimensional network (e.g. the repeat units are linked together left, right, up, and down in a sheet form), or a one-dimensional network (e.g. the repeat units are linked left and right to form a chain). “Polymers” can be composed, natural monomers or synthetic monomers and combinations thereof. The polymers can be biologic (e.g. the monomers are biologically important (e.g. an amino acid), natural, or synthetic.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as Port Wine Birthmarks or attendant issues. The effect can be therapeutic in terms of a partial or complete cure of a disease, condition, symptom or adverse effect attributed to the disease, disorder, or condition. The term “treatment” as used herein covers any treatment of Port Wine Birthmarks and attendant issues, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatments alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “water-soluble”, as used herein, generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

All patents, patent applications, published applications, and publications, databases, websites and other published materials cited herein are hereby incorporated by reference to the same extent as though each individual publication, published patent document, or patent application was specifically and individually indicated as being incorporated by reference.

### Kits

Any of the methods for identifying and characterizing candidate molecules described herein can be presented as a combination kit. As used herein, the terms “combination kit” or “kit of parts” refers to the components, parts, pieces, modules, and any additional components that are used to package, sell, market, deliver, and/or provide the combination of elements or a single element, such as the methods for identifying and characterizing candidate molecules described herein. Such additional components include, but are not limited to, packaging, blister packages, and the like. When one or more of the components, parts, pieces, modules, and any additional components described herein or a combination thereof (e.g., a method for identifying and characterizing candidate molecules provided alone or a method for identifying and characterizing candidate molecules provided with constituent parts/pieces for installation) contained in the kit are provided simultaneously, the combination kit can contain the methods for identifying and characterizing candidate molecules alone or the methods for identifying and characterizing candidate molecules provided with other accoutrements for installation, modification, and/or upkeep. When the components, parts, pieces, modules, and any additional components described herein or a combination thereof and/or kit components are not provided simultaneously, the combination kit can contain the methods for identifying and characterizing candidate molecules and constituent parts in separate combinations. The separate kit components can be contained in a single package or in separate packages within the kit.

In some embodiments, the combination kit also includes instructions printed on or otherwise contained in a tangible medium of expression. The instructions can provide information regarding the methods for identifying and characterizing candidate e molecules, installation/upkeep/maintenance information, information regarding use, etc. In some embodiments, the instructions can provide directions and protocols for using/operating the methods for identifying and characterizing candidate molecules or providing maintenance to same. In some embodiments, the instructions can provide one or more embodiments of the methods for using the identifying and characterizing candidate molecules methods of the current disclosure as any of the methods described in greater detail elsewhere herein.

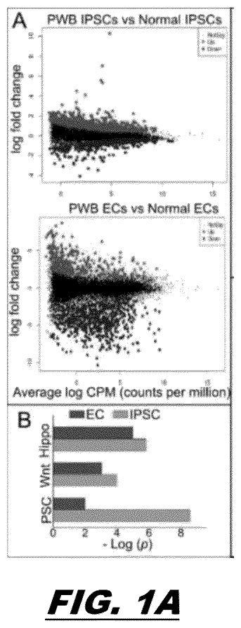

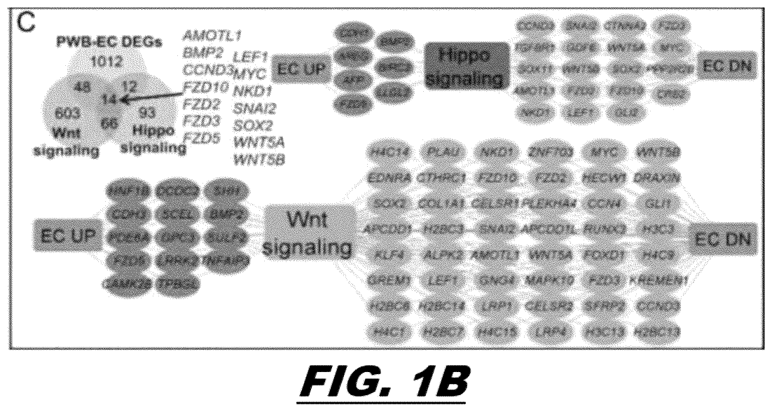

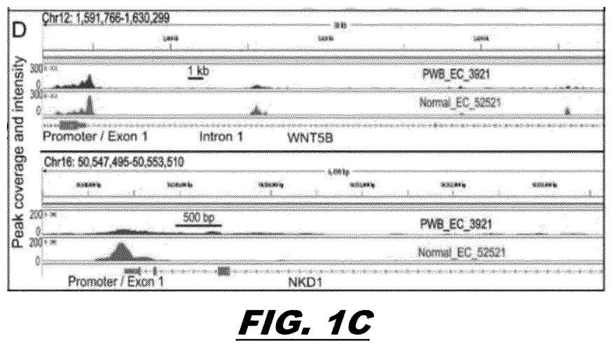

The current disclosure analyzed transcriptomes and pathway signatures of PWB iPSCs. A “PWB iPSC” refers to an “induced pluripotent stem cell” (iPSC) derived from a patient with a “port wine birthmark” (PWB), meaning the stem cell is created by reprogramming skin cells taken from a lesion on a person with this type of vascular malformation, allowing researchers to study the disease in a lab setting by differentiating the iPSCs into relevant cell types like endothelial cells (ECs) and ECs versus normal ones. The inventors performed bulk RNA sequencing (RNA-seq) and transposase accessible chromatin with sequencing (ATAC-Seq) on the iPSCs and their derived ECs compared with their counterparts to reveal the differentially expressed genes (DEGs). (FIG. 1A, false discovery rate (FDR) <0.01 as the cutoff threshold).

FIGS. 1A-1C show (A) Transcriptome profiles of PWB iPSCs and ECs versus normal ones (n=6-8 for each group). RNA-seq data plot showing significantly upregulated (red dots) or downregulated (blue dots) genes (FDR<0.01). Black dots: genes without expression changes. (B) KEGG analysis of RNA-seq DEGs showing significantly dysregulated pathways overlapped in both PWB iPSCs and ECs as compared with normal ones. −log (P) was plotted. (C) Enriched DEGs associated with Hippo and Wnt signalosomes in PWB iPSC-derived EC from bulk RNA-seq data. (D) The coverage and intensity of called peaks showing more chromatin accessibility in the promoters of WNT5B and NKD1 genes in PWB ECs as compared to normal ECs using ATAC-seq.

Kyoto Encyclopedia of Genes and Genomes (KEGG) annotation analyses showed that regulating pluripotent stem cells (PSC), Wnt and Hippo pathways were the significantly downregulated pathways present in both PWB iPSCs and ECs compared to normal iPSCs and ECs, respectively (FIG. 1A).

The Hippo and Wnt signaling pathways are signaling cascades that control cell proliferation, differentiation, and apoptosis. They are important for maintaining tissue homeostasis and organ size.

### Hippo Pathway

A cascade of kinases that controls the phosphorylation of YAP and TAZ, which are transcriptional coactivators. The Hippo pathway prevents YAP and TAZ from translocating to the nucleus, where they induce the transcription of genes involved in cell proliferation.

### Wnt Pathway

A family of glycoproteins that activate three different pathways: the canonical Wnt/β-catenin cascade, the noncanonical planar cell polarity (PCP) pathway, and the Wnt/Ca²⁺ pathway.

There were 14 overlapped DEGs in ECs in both pathways (FIG. 1B). The bulk ATACseq data demonstrated that many DEGs (such as WNT5B and NKD1) associated with both Hippo and Wnt pathways showed higher chromatin accessibilities in normal ECs than PWB ECs (FIG. 1C), which were consistent with the bulk RNA-seq data (FIG. 1B). The Hippo pathway plays a fundamental role in organ size control, stem cell proliferation, and tissue repair and regeneration; while Wnt pathway conveys the crucial signaling to regulate cell fate determination, migration, polarity, and organogenesis, etc. The crosstalk between both signalosomes coordinately executes their functions during development. As the clinical manifestations of developmental vascular defects and soft tissue outgrowth in PWB, it is reasonable to posit the critical roles of impaired Hippo and Wnt pathways in development and progression of PWB lesional vascular phenotypes.

To discover candidate therapeutics for PWB through data-driven and deep learning-powered approaches Drug repurposing is a strategy to identify new applications for an existing drug beyond its original medical indication. These compounds have available preclinical and clinical data thus are de-risked with the advantages of low costs and short timelines. It will particularly benefit patients with rare and neglected or uncommon diseases, including PWB that are largely ignored by the pharmaceutical industries and currently lack effective treatments.

The inadequate clinical outcomes of current energy-based treatment modalities for PWB need paradigm-shifting therapeutics, such as targeting causal mechanistic signaling pathways to reduce lesional vascular phenotypes. This disclosure provides a strong rationale for introducing such a modality. We have shown that aberrant Hippo/Wnt pathways are related to the resulted PWB ECs.

### Experiments

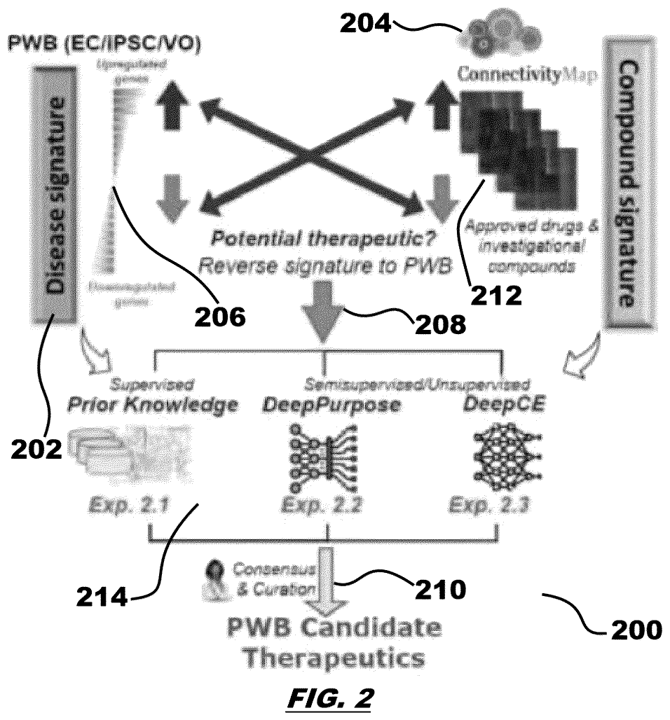

We used a use a data-driven computational approach 200 to identify candidate molecules and characterized them using prior knowledge of the PWB-associated biological processes and pathways or disease signature 202. The signature-based connectivity map (CMap)47 computational drug discovery module 204 took a collection of transcriptomic signatures or differentially expressed genes (DEGs) 206 as input 208 and outputs 210 a list of ranked drugs 212 predicted 214 to consistently reverse the input gene signature(s) as input. Using the PWB signature (DEGs), we queried large-scale perturbagen signatures (1.4 million L1000 profiles) from the NIH's Library of Integrated Network-based Cellular Signatures to identify small molecules that could be of therapeutic benefit in PWB (FIG. 2). The LINCS datasets include more than 20 K small-molecule compounds, including approved and investigational therapeutics. The LINCS Cloud query API46 and Kolmogorov-Smirnov-test (KS test) based algorithm was used to rank candidate compounds.

The pattern-matching software searched for two-directional matches, considering both the up and down query gene sets (PWB signature), in comparing the query against signatures (z-scored differential expressions) in the LINCS L1000 dataset (FIG. 2). The system then generated a list of signatures rank-ordered by the strength of the match to the query. To deduce the potential mechanism of action (MOA) of identified hits, we use a comprehensive perturbagen-induced proteome fingerprint atlas. Since, compounds with overlapping targets and MOAs typically elicited similar protein-level responses, we compared PWB-based compound hits to perturbagen-induced proteome fingerprints to deduce the MOA.

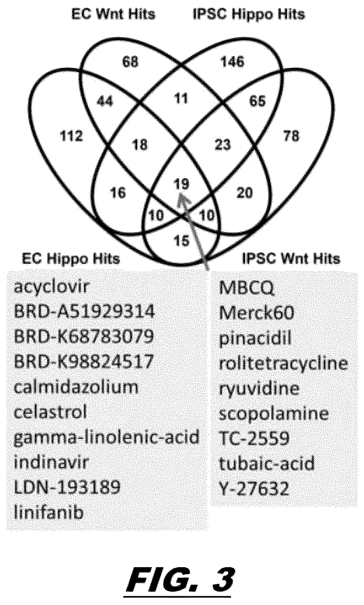

In this study, using transcriptomic data from the PWB ECs and iPSCs, we identified computationally several compounds that could reverse the PWB signature. Since, our focus was on differentiation-related signalosomes, specifically used Hippo and Wnt pathway genes that were dysregulated in PWB EC and iPSCs. We, initially, found 19 common compounds (FIG. 3) and among these were some compounds that could be potentially interesting such as celastrol, a natural pentacyclic triterpene compound. It is associated with vascular remodeling through inhibition of vascular inflammation, abnormal proliferation and migration of vascular smooth muscle cells, vascular calcification, endothelial-to-mesenchymal transition, ECM remodeling and angiogenesis. Interestingly, celastrol has also been reported to inhibit laser-induced choroidal neovascularization by decreasing VEGF induced proliferation and migration. The limited outcomes of current paradigm of PDL treatment in PWB is attributed to the regeneration and revascularization of the vascular structures after laser irradiation. Hence, the suppression of regeneration and revascularization of photocoagulated blood vessels with compounds such as celastrol can achieve an improved therapeutic outcome in patients with PWB, which is currently under active investigation in the inventor's lab.

Using an encoder-decoder framework to prioritize drug-target interactions.

We will use DeepPurpose, a powerful deep learning Python library, to prioritize the most likely drug candidates identified from our computational screening approaches, as described previously. DeepPurpose processes the input target amino acid sequences and candidate drug codes by feeding them into multiple latest deep learning models pre-trained on DAVIS, BindingDB-Kd, and kinase inhibitor bioactivity (KIBA) datasets. DeepPurpose presents the drug-target interaction (DTI) model as an encoder-decoder framework where molecular encoders are used to map the compounds and protein targets into vector representations, which are in turn used to predict DTIs. Taking the simplified molecular-input line-entry system (SMILES) format of the drug and the target amino acid sequence pair as inputs, DeepPurpose outputs the binding affinity scores between the drug and the target.

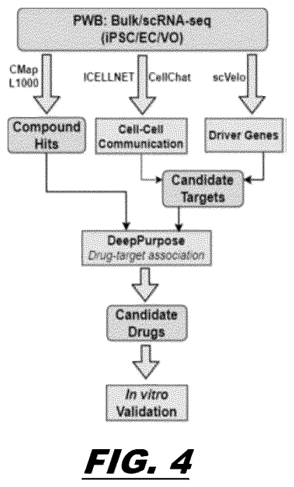

For drug molecules, DeepPurpose provides 15 encoders: Morgan, PubChem, Daylight, RDKit 2D, convolutional neural network (CNN), convolutional recurrent neural network (CNN+RNN), Transformer encoder, Message-Passing Neural Network (MPNN), and five different graph neural network (GNN) based compound encoding models. For protein targets, DeepPurpose provides 8 encoders: amino acid composition (AAC), PseudoAAC, Conjoint Triad, Quasi Sequence, CNN, CNN+RNN, and Transformer. Additionally, DeepPurpose also provides more than 10 pre-trained models trained on multiple DTI datasets. To use this library, we will first translate the target proteins into amino acid sequences and the potential drugs (E.g., from L1000 CMap analysis and/or other small molecule libraries) into SMILES fingerprints. Taking the sequences and fingerprints as input, binding affinity is identified and aggregated from multiple DeepPurpose pre-trained models, between each paired drug molecule drug molecule and protein target of interest (FIG. 4). Based on these aggregated binding affinity scores, with the top hits selected for experimental validation. FIG. 4 shows a schematic representation of encoder-decoder framework to discover and prioritize drug target interactions.

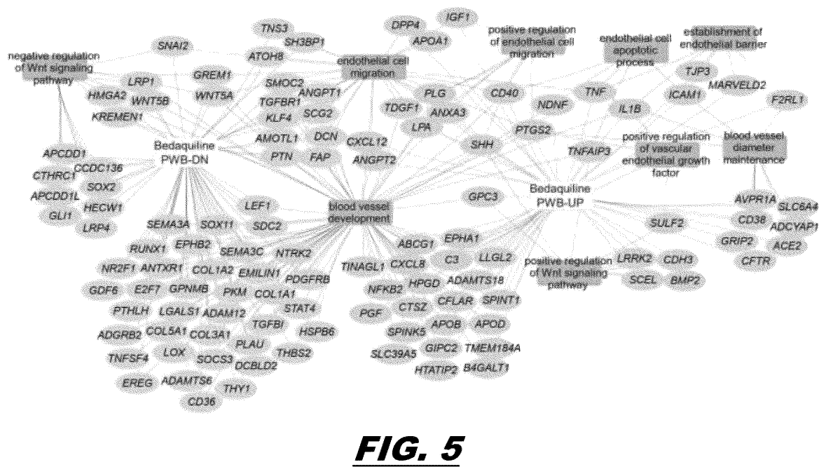

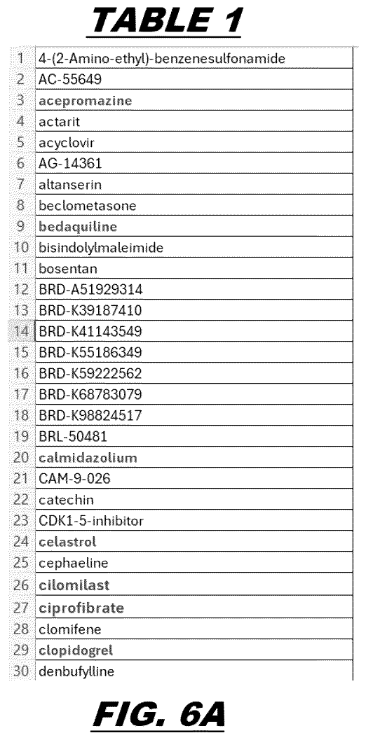

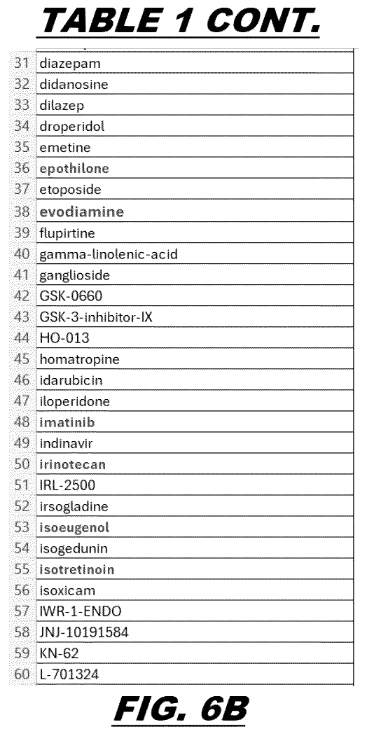

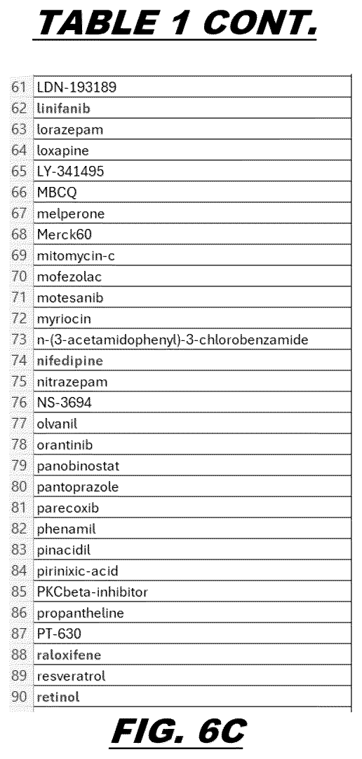

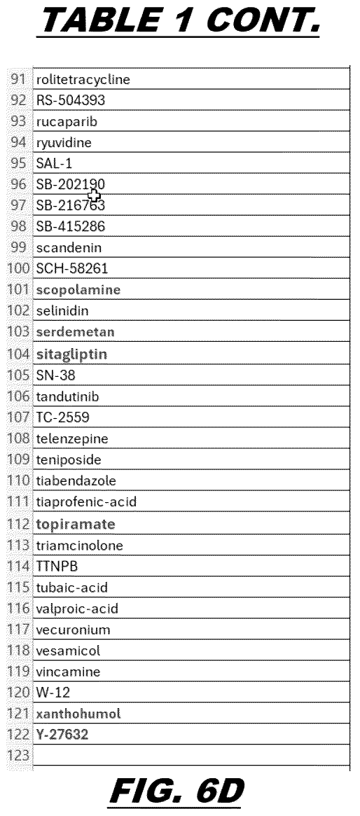

As part of the preliminary analysis, using the DEGs from PWB ECs and our implemented DeepPurpose pipeline, we identified several compounds. Among these bedaquiline (Sirturo), an FDA-approved drug to treat active tuberculosis, was the top hit with high binding affinity predicted for several of the proteins encoded from PWB EC DEGs (FIG. 5). To characterize bedaquiline and to understand its potential MOA with respect to PWB, we performed a functional enrichment analysis with our ToppFun application, using DeepPurpose-predicted bedaquiline targets that were differentially regulated in PWB ECs. Among the top enriched biological processes were blood vessel development, EC migration, blood vessel diameter maintenance, and Wnt regulation.

Interestingly, bedaquiline is reported to inhibit capillary network formation of human lung tumor associated endothelial cell on matrigel and its multiple functions, such as spreading, proliferation and apoptosis, even in the presence of vascular endothelial growth factor (VEGF). Bedaquiline is also reported to activate host macrophage innate immune resistance to bacterial infection, and macrophages play major roles in many angiogenesis models by secreting several inflammatory and angiogenic mediators67. Bedaquiline was the top hit for PWB iPSCs too suggesting its repurposing potential for PWB.

Table 6 shows further compounds determined as suitable for the treatments and methods described herein including: 4-(2-Amino-ethyl)-benzenesulfonamide, AC-55649, acepromazine, actarit, acyclovir, AG-14361, altanserin, beclomethasone, bedaquiline, bisindolylmaleimide, bosentan, BRD-A51929314, BRD-K39187410, BRD-K41143549, BRD-K55186349, BRD-K59222562, BRD-K68783079, BRD-K98824517, BRL-50481, calmidazolium, CAM-9-026, catechin, CDK1-5-inhibitor, celastrol, cephaeline, cilomilast, ciprofibrate, clomifene, clopidogrel, denbufylline, diazepam, didanosine, dilazep, droperidol, emetine, epothilone, etoposide, evodiamine, flupirtine, gamma-linolenic-acid, ganglioside, GSK-0660, GSK-3-inhibitor-IX, HO-013, homatropine, idarubicin, iloperidone, imatinib, indinavir, irinotecan, IRL-2500, irsogladine, isoeugenol, isogedunin, isotretinoin, isoxicam, IWR-1-ENDO, JNJ-10191584, KN-62, L-701324, LDN-193189, linifanib, lorazepam, loxapine, LY-341495, MBCQ, melperone, Merck60, mitomycin-c, mofezolac, motesanib, myriocin, n-(3-acetamidophenyl)-3-chlorobenzamide, nifedipine, nitrazepam, NS-3694, olvanil, orantinib, Panobinostat, pantoprazole, parecoxib, phenamil, pinacidil, pirinixic-acid, PKCbeta-inhibitor, propantheline, PT-630, raloxifene, resveratrol, retinol, rolitetracycline, RS-504393, rucaparib, ryuvidine, SAL-1, SB-202190, SB-216763, SB-415286, scandenin, SCH-58261, scopolamine, selinidin, serdemetan, sitagliptin, SN-38, tandutinib, TC-2559, telenzepine, teniposide, tiabendazole, tiaprofenic-acid, topiramate, triamcinolone, TTNPB, tubaic-acid, valproic-acid, vecuronium, vesamicol, vincamine, W-12, xanthohumol, Y-27632, and/or combinations of the above.

Preferred compounds include but are not limited to acepromazine, bedaquiline, calmidazolium, celastrol, cilomilast, ciprofibrate, clopidogrel, epothilone, evodiamine, imatinib, irinotecan, isoeugenol, isotretinoin, linifanib, nifedipine, raloxifene, retinol, scopolamine, serdemetan, sitagliptin, topiramate, xanthohumol, Y-27632, and/or combinations of the above.

Further embodiments are illustrated in the following Examples which are given for illustrative purposes only and are not intended to limit the scope of the disclosure.

Various modifications and variations of the described methods, compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A method for repurposing an existing drug to treat Port Wine Birthmarks comprising:
employing at least one data-driven computational module to identify and characterize at least one existing drug candidate molecule based on at least one effect of the at least one existing drug candidate on at least one Port Wine Birthmark biological process;
inputting at least one transcriptomic signature as at least one input; and
outputting at least one existing drug, wherein the at least one existing drug is selected to reverse the at least one transcriptomic signature used as the at least one input.

2. The method for repurposing an existing drug to treat Port Wine Birthmarks of claim 1, wherein an original medical indication of the existing drug does not include treatment of Port Wine Birthmarks.

3. The method for repurposing an existing drug to treat Port Wine Birthmarks of claim 1, wherein the existing drug targets at least one causal mechanistic signaling pathway to reduce at least one lesional vascular phenotype.

4. The method for repurposing an existing drug to treat Port Wine Birthmarks of claim 1, wherein the at least one causal mechanistic signaling pathway comprises at least one aberrant Hippo/Wnt pathway.

5. The method for repurposing an existing drug to treat Port Wine Birthmarks of claim 1, wherein the existing drug is selected from acepromazine, bedaquiline, calmidazolium, celastrol, cilomilast, ciprofibrate, clopidogrel, epothilone, evodiamine, imatinib, irinotecan, isoeugenol, isotretinoin, linifanib, nifedipine, raloxifene, retinol, scopolamine, serdemetan, sitagliptin, topiramate, xanthohumol, Y-27632 and/or combinations of the above.

6. The method for repurposing an existing drug to treat Port Wine Birthmarks of claim 1, wherein the at least one data-driven computational module comprises a signature-based connectivity map (CMap)47 computational drug discovery module.

7. The method for repurposing an existing drug to treat Port Wine Birthmarks of claim 1, wherein multiple existing drugs are provided as an output.

8. The method for repurposing an existing drug to treat Port Wine Birthmarks of claim 1, wherein the multiple existing drugs provided as an output are ranked in order of efficacy with respect to reversing the at least one transcriptomic signature used as the at least one input.

9. The method for repurposing an existing drug to treat Port Wine Birthmarks of claim 8, wherein an encoder-decoder framework is used to rank the multiple existing drugs provided as the output via presenting at least one predictive drug-target interaction model.

10. The method for repurposing an existing drug to treat Port Wine Birthmarks of claim 1, wherein at least one functional enrichment analysis is conducted on the at least one existing drug candidate to characterize the at least one existing drug candidate with respect to blood vessel development, EC migration, blood vessel diameter maintenance, and Wnt regulation.
